(19) United States
(12) Patent Application Publication
(10) Pub. No.: US 2003/0105045 A1
(43) Pub. Date: Jun. 5, 2003

(54) Artificial transcriptional factors and methods of use

(75) Inventor: Dusan Stanojevic, Arlington, MA (US)

Correspondence Address: HALE AND DORR, LLP, 60 STATE STREET, BOSTON, MA, 02109

(73) Assignee: Crosslink Genetics Corporation, Cambridge, MA (US)

(21) Appl. No.: 10/097,800

(22) Filed: Mar. 14, 2002

Related U.S. Application Data
(63) Continuation of application No. 09/977,852, filed on Oct. 15, 2001, now abandoned.
(60) Provisional application No. 60/240,479, filed on Oct. 13, 2000.

(51) Int. Cl. A61K 48/00; C12N 5/00; C12N 15/85

(52) U.S. Cl. 514/44; 435/455; 435/375

(57) ABSTRACT

A description of artificial transcription factors (ATFs) is provided having a non-peptidic-DNA binding domain, flexible linker and a short synthetic effector domain. The ATFs are highly potent transcriptional modulators in vitro and in vivo. Methods for targeted manipulation of gene expression and the development of new class of pharmaceuticals are also provided.

### CROSS-REFERENCE TO RELATED APPLICATIONS

The application claims priority under 35 U.S.C. §119(e) from provisionally filed application U.S. S. No. 60/240,479, filed on Oct. 13, 2000, and entitled “Artificial Transcriptional Factors and Methods of Use.”

This application is a continuing application of co-pending application Ser. No. 09/977,852, filed on Oct. 15, 2001, entitled “Artificial Transcriptional Factors and Methods of Use”.

### GOVERNMENT FUNDING

This invention was made in part with government support under grant 1R41 GM57712-01 awarded to the inventor by the National Institute of General Medical Sciences. The government has certain rights in the invention.

### BACKGROUND OF THE INVENTION

This invention relates to regulation of gene expression, and in particular, to the design and synthesis of compositions useful as artificial transcriptional factors (ATFs). The invention also relates to the use of such molecules as novel therapeutics that regulate gene expression at the level of RNA transcription, as tools for gene regulation and for target validation in functional genomics, and in pharmaceutical drug development.

A crucial step in eukaryotic genome regulation involves activation and repression of RNA synthesis by transcription factors. Transcription factors are modular proteins that contain at least two functional parts: a DNA-binding domain and an activation or repression (i.e. “effector”) domain (Ptashne, M. “A Genetic Switch: Phage Lambda and Higher Organisms” Cell and Blackwell Scientific, Cambridge, Mass., (1992); and Ptashne, M. et al., Nature 386: 569-577 (1997)). Typically, the DNA-binding domain anchors the transcription factor to the promoter through interaction with specific DNA sequences. Effector domains participate in interactions with other proteins involved in RNA transcription such as TATA-BOX binding protein (TBP) and TBP-associated proteins, proteins comprising RNA Polymerase II holoenzyme, coactivators, corepressors, histones and histone-modifying enzymes, and others (Blau, J. et al., Mol. Cell Biol. 16: 2044-2055 (1996); Brown, S. A. et al., EMBO J 17: 3146-3154 (1998); Collingwood, T. N. et al., J. Mol. Endocrinol. 23: 255-275 (1999)). These protein-DNA and protein-protein interactions facilitate or inhibit the assembly of the transcriptional apparatus at the promoter and, therefore, activate or repress the synthesis of RNA encoded by the regulated gene (Koleske, A. et al., Trends. Biochem. Sci. 20: 113-116 (1995): Hampsey, M. and Reinberg, D. Curr. Opin. Genet. Dev. 9: 132-139 (1999)).

Experimental data reported thus far strongly indicates that effector domains are functionally independent from DNA-binding domains. For example, an activation domain of one transcription factor can be attached to the DNA-binding domain of a different factor. Such hybrid molecules retain their characteristic functions across the range of organisms, from yeast to human cells, thus indicating that the basic regulatory mechanisms are common to diverse eukaryotes (Brent, R. et al., Cell 43: 729-736 (1985)). Therefore, the DNA-binding and effector domains could be considered as completely separate functional and structural entities joined together as a bifunctional molecule.

There have been several recent attempts to combine the minimal activation domain of strong transcriptional activators like VP16 or GCN4 with artificial DNA-binding domains based on a triple-helix forming oligonucleotide or a pyrrole oligomers. These efforts have yielded molecules having a barely detectable activity (Kuznetsova, S. et al., Nucleic. Acids Res. 27: 3995-4000 (1999)) or showing only a weak activation limited to in vitro (cell-free) assays (Mapp, A. K. et al., Proc. Natl. Acad. Sci. USA 97: 3930-3935 (2000)). In general, the activity of such hybrid molecules is extremely weak (and possibly non-specific) compared to that of the natural (or protein) activators from which portions of these molecules were derived (Sadowski, I. et al., Nature 335: 563-564 (1988)).

There remains a need for the identification of novel transcription activators and improved activation systems. In particular, there remains a need for artificial transcription activators that match or exceed the activation of strong natural transcriptional activators. There also remains a need for novel transcription repressors and repressor systems. In general, there remains a need for novel transcription systems that can modulate the transcription process. The molecules would open the possibility for the first practical applications of the technology in biology and medicine.

### SUMMARY OF THE INVENTION

The present invention relates to a molecular design resulting in highly potent ATFs, as is demonstrated both in cell-free assays (in vitro) and in vivo (in tissue culture cells). These ATFs may be used as tools for gene regulation at the level of transcription and as a class of gene targeting pharmaceuticals for treatment of cancer and many other diseases.

Conceptually, the modular structure of transcription factors implies that each transcription factor molecule could be simply regarded as a bi-dentate ligand, where one of the ligands (DNA-binding domain) binds DNA in a sequence-specific manner and other ligand (the “effector” domain) binds a protein that participates in transcriptional regulation (activation or repression). This simplified model raises a possibility of replacing the DNA-binding and/or effector domain with synthetic chemical moieties to yield a novel class of molecules—synthetic or artificial transcription factors (ATFs). The main goal of the ATF molecular design is to introduce new, drug-like chemical properties such as a lower molecular weight, resistance to enzymatic degradation and cell membrane permeability while preserving the crucially important biological function—the ability to regulate RNA transcription of specific genes.

In one aspect of the invention, a composition for modulating transcription of a eukaryotic gene is provided. The composition includes a non-peptidic DNA binding domain, a flexible linker, and a transcriptional effector, one end of the linker being covalently bound to the DNA binding domain, and the other end of the linker being covalently bound to the transcriptional effector. In at least some embodiment, the flexible linker has a length in the range of 10-100 Å. In at least some embodiments, the flexible linker has a length in the range of 15-25 Å, or in the range of 25-40 Å, or in the range of 40-60 Å, or in the range of 60-100 Å.

In at least some embodiments, the composition for modulating transcription of a eukaryotic gene binds a co-modulating protein. In at least some embodiments, the composition represses transcription and binds a histone or histone modifying protein.

In at least some embodiments, the DNA binding domain is a nucleic acid. In at least some embodiments, the nucleic acid is a modified nucleic acid. In another embodiment, the nucleic acid includes a modified backbone, or the nucleic acid contains modified bases. In still other embodiments, the modified backbone comprises substitutions for phosphodiester bonds selected from the group consisting of phosphorothioates and peptide nucleic acids. In other embodiments, the linker is bound to the 3′ or the 5′end of the nucleic acid.

In at least some embodiments, the DNA binding domain is a peptidic nucleic acid. In some embodiments, the DNA binding domain does not contain a plurality of pyrrole or imidazole groups. In at least some embodiments the DNA binding domain is a sequence-specific DNA-binding natural product.

In at least some embodiments, the transcriptional effector is a polypeptide sequence. In some embodiments, the transcriptional effector is a polypeptide activator and the polypeptide has a polypeptide sequence including at least one copy of an activator sequence of amino acids from Herpes simplex viral protein VP16 comprising SEQ ID NO:1 (see, Table 1.) (GSDALDDFDLD). In at least one embodiment, the polypeptide has the sequence of SEQ ID NO:12. In at least some embodiments, the peptide sequence includes two copies of the activator sequence of SEQ ID NO:1 and a cysteine residue at an amino terminus of the peptide sequence, the peptide having the sequence of SEQ ID NO: 2 (see, Table 1.) (CGSDALDDFDLDGSDALDDFDLD). In at least some embodiments, the polypeptide sequence includes a cysteine residue and two copies of the activator sequence SEQ ID NO:12, the peptide having the sequence of SEQ ID NO:8. In at least some embodiments, the amino-terminus of the polypeptide transcriptional effector is covalently bound to the linker. In at least some embodiments, the carboxyl-terminus of the polypeptide transcriptional effector is covalently bound to the linker. In at least some embodiments, a cysteine residue of the polypeptide transcriptional effector is covalently bound to the linker.

In at least some embodiments, the transcriptional effector moiety of the composition for modulating transcription of a eukaryotic gene has a molecular weight of less than about 3,000 daltons. In at least some other embodiments, the transcriptional effector moiety has a molecular weight of less than about 1,500 daltons. In at least some other embodiments, the transcriptional effector moiety has a molecular weight of less than about 1,000 daltons.

In at least some embodiments of the composition for modulating transcription of a eukaryotic gene, the flexible linker includes a polyglycol. In some embodiments, the flexible linker polyglycol contains at least two, or at least four, or at least six, glycol units. In another embodiment, the flexible linker includes a plurality of monomer units selected from the group consisting of nucleotides, peptides, and lower alkyls or other oxygen containing alkyl chain derivatives.

In at least some embodiments, the amount of transcription initiated on a double-stranded DNA template is at least ten-fold greater compared to a second amount initiated in the absence of the composition of the invention. In at least some embodiments, the amount of transcription initiated on a linear double-stranded DNA template is at least 20-, or at least 30-, or at least 40-, or at least 50-fold greater compared to the second amount in the absence of the composition of the invention. In at least some embodiments, the amount of transcription is 30-50 times greater than transcription in the absence of the ATF of the present invention.

In at least some embodiments of the invention, the DNA binding domain of the compositions for effecting (modulating) transcription of a eukaryotic gene has affinity for at least one DNA binding site on a DNA template, which DNA template is less than about 500 base pairs in length.

In another aspect of the invention, a composition for activating transcription has the structure A-B-C, wherein A is a triplex-forming nucleic acid, B is a flexible linker, and C is an activation moiety that binds to a site on a transcriptional protein complex comprising an eukaryotic RNA polymerase, wherein B is covalently linked to A and C. In at least some embodiments, B is a polyglycol chain, and the covalent linkage of B to C includes a bifunctional crosslinking agent. In at least some embodiments, the linker, B, is a polyglycol of at least about 28 Å in length, and C comprises an amino acid sequence from Herpes simplex viral protein VP16.

Another aspect of the invention provides a method for assaying a test compound for activity as a transcriptional effector The method includes linking the test compound covalently to a flexible linker domain which is covalently bound to a non-peptidic DNA binding domain to provide a test composition, the DNA binding domain having affinity for a DNA binding site on a DNA template sufficient to bind the site and to modulate transcription at a promoter; contacting the test composition with a transcription mixture including a DNA template, a eukaryotic RNA polymerase molecule capable of forming a complex with the test composition and the DNA template, a buffer and substrates under conditions suitable for RNA synthesis, such that RNA is synthesized; and determining the quantity of RNA produced in the presence of the test composition compared to a level in the absence of the test composition, which is a measure of the activity of the test composition as a transcriptional modulator. In at least some embodiments, the DNA binding site is a plurality of repeats of the binding site sequence. In at least some embodiments of the method for assaying a test composition for activity as a transcriptional activator, the binding site of the test composition to the DNA template is located within 100 base pairs of the site for initiation of transcription.

In another embodiment of the method for assaying a test composition for activity as a transcriptional activator, the step of providing the test composition with a transcription mixture is performed in vivo. In at least some embodiments, the test composition is pre-bound to the template, and the complex is provided to a plurality of cells by transformation. In at least some embodiments, the test composition is provided to the cells that carry a reporter plasmid template incorporated into the chromosome (stable transfection lines).

In at least some embodiments, the step of providing the test composition with a transcription mixture is performed using high throughput screening technologies comprising robotized distribution into wells of multiwell dishes. In at least some embodiments, the step of determining the quantity of transcription is performed using high throughput methods of detection comprising automated plate readers having programmable computerized programs for data analysis and display.

### BRIEF DESCRIPTION OF THE DRAWINGS

FIG. 1 shows a pictorial illustration of the tripartite ATF of the invention. The ATF has three component parts, a DNA binding domain (designated A), a flexible linker (B), and an effector domain, for example, an activator domain (C).

FIG. 2 shows (A) an ATF of the present invention and (B) one possible scheme for synthesis of an ATF in at least some embodiments of the invention.

FIG. 3 shows the promoter regions of the transcription templates, wherein: (A) the control template contains in promoter five GAL4 and five ATF binding sites incorporated in the promoter at −53 and −155 bp relative to the +1 transcription start site, respectively; and (B) the ATF assay template contains five ATF binding sites incorporated at −65 bp.

FIG. 4 shows an acrylamide gel electrophoretogram of the run-off transcription products in the presence and absence of an ATF (as described in FIG. 2A), with the contents of each lane identified as follows: lane 1 shows the low level of basal transcription from the control transcription template of FIG. 2A; lane 2 shows the 250 base transcript activated by the GAL4-VP16 fusion protein on the control template; lane 3 shows basal transcription from the ATF assay transcription template of FIG. 2B; lanes 4-7 show the effect of the ATF of FIG. 1 on transcription from the ATF assay transcription template, with lane 4 having the smallest amount of ATF, and lane 5 having five-fold more ATF than lane 4, lane 6 having 100-fold more ATF than lane 4, and lane 7 having 500-fold more ATF than lane 4; lanes 8 and 9 show the effect of an ATF having the polylinker, with covalently attached activation domain, the polylinker being attached to the 3′ terminus of the DNA binding domain, on transcription of the ATF test template, where lane 9 has twenty-fold more ATF than lane 8.

FIG. 5 shows an acrylamide gel electrophoetagram of the run-off transcription products for transcriptional activators GAL4-VP16, 3′ ATF, 5′ ATF(D) and 3′ ATF(D).

FIG. 6 shows (A) reporter constructs used in cotransfection assays, in which the transcription template contains five ATF binding sites in the promoter, (B) a representative CAT assay whereby BHK-21 were cotransfected with transcription template alone (lanes 1 and 2) or in combination with ATF's (lanes 3-6), and (C) a bar graph of the mean fold transcriptional activation by ATFs for three experiments, each performed in duplicate; error bars represent SEM (n=3).

### DETAILED DESCRIPTION OF THE INVENTION

### Definitions

The following definitions are used throughout this description and in the claims, unless the context otherwise requires. The patents and scientific literature referred to herein establishes the knowledge that is available to those of skill in the art. The issued U.S. patents, allowed applications, and references cited herein are hereby incorporated by reference.

As used herein the term “artificial transcription factors” includes synthetic transcription factors.

The term “about” is used herein to mean a numeral value having a range of ±20% around the cited value.

The term “transformation” refers to a genetic event used in construction of a cell line or strain, resulting from mixing recipient cells with DNA such that the DNA enters at least a portion of the cells, and includes transfection, lipofection or other liposome-mediated process, and electroporation.

A “transcriptional effector” refers to a chemical composition which when present in the vicinity of a promoter, and bound to a DNA binding domain, causes an increase or decrease in quantity of RNA synthesized from a particular promoter or class of promoters. Transcription in the absence of an effector is said to be at a “basal” level. Transcription can be activated (also known as induced, or up-regulated) by a positive effector or “activator”. Similarly, a basal or activated level can be repressed, or down-regulated, by a negative effector or “repressor”.

A transcriptional effector can act near or at the site of initiation of transcription of a gene, or over a distance. Genes are transcribed when RNA is synthesized in a 5′ to 3′ direction using a strand of DNA as a template. The site of initiation of transcription, in which a first ribonucleoside triphosphate complexes with the RNA polymerase, occurs complementary to a site on the DNA template known as “+1”, with each successive nucleotide addition occurring complementary to “downstream” sites with increasing positive cumbers. “Upstream” of the +1 site are generally found the DNA regulatory signals, such as the promoter, that specify binding of the transcription factors, components of the transcriptional machinery, RNA polymerase and associated proteins. A promoter is generally found within a fixed distance upstream of the +1 site, to position the RNA polymerase holoenzyme appropriately for transcription initiation. Regulatory signals in the DNA sequence that modulate the amount of transcription, such as the GAL-4 binding site sequence originally discovered in yeast, are generally located in the promoter, for example, upstream of or adjacent to the +1 site.

Nucleotide and amino acid sequences referred to herein are listed in Table 1.

| SEQ ID NO | Sequence |
|---|---|
| 1 | NH₂-GSDALDDFDLD-COOH |
| 2 | NH₂-CGSDALDDFDLDGSDALDDFDLD-COOH |
| 3 | NH₂-FLFQLPQQTQGALLTSQP-COOH |
| 4 | NH₂-WAVYELLF-COOH |
| 5 | 5′TTGTGGTGGGTGGGGTGTGGGT3′ |
| 6 | 5′TTGTGGTGGGTGGGGTGTGGGTXYC3′ |
| 7 | 5′CYXCTTGTGGTGGGTGGGGTGTGGGT3′ |
| 8 | NH₂-CGSDALDDFDLDMLGSDALDDFDLDMLGS-COOH |
| 9 | 5′GTTCTCCTCCCTCCCCTCTCCCTCTT3′ |
| 10 | 3′AAGAGGAGGGAGGGGAGAGGGAGAAC5′ |
| 11 | 5′TTCTCCTCCCTCCCCTCTCCCTCTT3′ |
| 12 | CGSDALDDFDLDML |

A means alanine; C means cysteine; D means aspartic acid; E means glutamic acid; F means phenylalanine; G means glycine; H means histidine; I means isoleucine; K means lysine; L means leucine; M means methionine; N means asparagine; P means proline; Q means glutamic acid; R means arginine; S means serine; T means threonine; V means valine; W means tryptophan; and Y means tyrosine.

### DETAILED DESCRIPTION OF EMBODIMENTS

It has been surprisingly discovered that a tripartite ATF, in which no part contains an intact, folded, protein domain, can function as well as or better in activating transcription than the protein transcription modulators from which the design of these portions are taken. The design of the tripartite molecule is such that the ATF contains a DNA binding domain, a flexible linker, and a transcriptional effector, that activates or represses or otherwise modifies transcription, also referred in the description and the claims as A, B, and C, respectively. None of these “domains,” however, need be a protein domain. The meaning of “domain,” as used herein, refers to the function of each of the three parts of the ATF molecule.

In one aspect of the invention, synthetic or artificial transcription factors (ATFs) are provided with activity comparable or even exceeding that of the natural transcriptional activator proteins to which certain ATF components are related. In some examples of the invention, the transcription response, i.e., either activation or repression, is 10 times, or 20 times, or 30 times or 40 times or even 50 times greater than that of the controls, i.e., a system in the absence of the ATF of the invention.

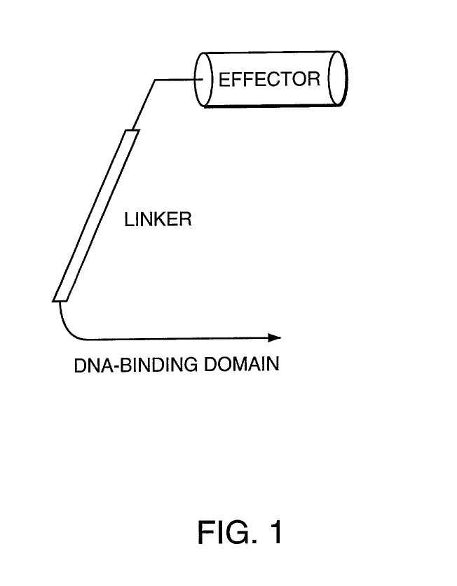

An embodiment of the ATF of the invention is illustrated in FIG. 1. The functional ATF includes a DNA binding domain A, shown here as a triplex forming oligonucleotide (TFO) bound at the 5′ position to a linker B. It is possible, however, to bind the linker at the 3′ end of the DNA binding domain with no loss in activity. The linker is joined at its distal end to the effector domain, C, i.e., a domain which has an effect on transcription. The effect may be to activate or repress transcription. Here, the effector is shown as an activating domain, AD. As is described in greater detail below, the individual domains of the ATF molecule are most typically covalently bound; however, it is contemplated that any appropriate association, i.e., covalent bonding, hydrogen bonding, hydrophilic or hydrophobic association, may be used to form the ATF.

The DNA binding domain, A, is any non-peptidic moiety with affinity for a specific recognition site within the promoter DNA. By non-peptidic moiety, it is meant that the domain does not include a substantial amount of a natural amino acid. Substantially excluding peptidic components in the DNA binding domain does not exclude the possibility of isolated inclusion of amino acids. For the purpose of this invention, substantially non-peptidic shall mean less than 50% or less than 20% of natural amino acid content. The choice of DNA binding domain depends on the gene intended to be activated. The DNA binding domain recognizes a site that is typically positioned relatively near to the transcriptional start site of the gene for which the activator can affect transcription, although some activators may be able to act over long distances. Many activators or repressors are able to act over long distances and use of these effectors is contemplated in the invention.

In at least some embodiments, the DNA binding domain is an oligonucleotide, such as a triplex forming oligonucleotide (TFO). These moieties are thought to bind in the major groove of the DNA helix. Design of triplex forming nucleic acids is described in U.S. Pat. No. 5,874,555 to Dervan, et al, which is incorporated herein by reference. It is anticipated that additional types of DNA binding domains can be substituted for triplex-forming nucleic acids.

TFOs are able to form strong and stable sequence-specific complexes with double-stranded DNA at physiological conditions (Maher, L. J. et al., Biochemistry 31: 70-81 (1992)). This property is not adversely affected upon conjugation with effectors, as is established in the examples below. Although not being bound to any mode or theory of operation, it is possible that the linker plays a role, among other possible roles of the linker, in “insulation” of the TFO from interference by the attached chemical groups.

The triple helix formation between the TFO and DNA is facilitated when sequences of both molecules have special features, most notably uniform stretches of purine bases in one DNA strand and pyrimidine in the complementary strand. This is not an obstacle for many applications such as pharmaceutical (gene targeting) applications or in vitro and in vivo ATF assays because the sequences on TFOs and its corresponding binding sites in any promoter or a reporter gene can be chosen at will. In addition, such sites have been shown to exist in native genes, most often in the regulatory regions of promoters (Svinarchuk, F. et al., Nucleic Acid Res. 22: 3742-3747 (1994); Nakanishi, M. et al., Nucleic Acid Res. 26: 5218-5222 (1998); Bramachari, S. K. et al., Gene 190: 17-26 (1997)). In addition, TFOs that are chemically modified may recognize many additional kinds of DNA sequences. For example, it is now possible to target a polypurine stretch interrupted by several pyrimidine residues with the “bridged” or clamped TFOs (Helene, C. et al., Ciba Found. Symp. 209: 94-102 (1997)). Also, the triplex recognition scheme can be extended by synthesizing TFOs with nonnatural bases and nucleotide analogs. TFO-based ATF may take advantage of the recent improvement and availability of genomic databases that makes it possible to identify convenient TFO-binding sites in promoters. In one application of the present invention, genes of medical interest and suitable binding sites are identified and a TFO sequence having a suitable affinity therefore is synthesized.

In addition to TFOs, there are other classes of molecules that can be used as artificial (non-peptidic) DNA-binding domains. In at least some embodiments of the present invention, the DNA binding domain is a peptide nucleic acid (PNA), a molecular analog of DNA in which the phosphate backbone is replaced with a backbone similar to that found in peptides. Peptide nucleic acids can bind to single-stranded DNA by Watson-Crick base pairing and can form triple helices to DNA/PNA duplexes much in the way of nucleosides (Nielsen, P. E. et al., J. Molec. Recognit. 7: 165-170 (1994); Egholm, M. et al., Nature 365: 566-568 (1993); and Kim, J. H. et al., Nucleic Acid Res. 27: 2842-2847 (1999)). A PNA “clamp” consisting of two PNA strands connected with a flexible linker can form a very stable complex with DNA duplex, and can be designed to target similar kind of polypurine and polypirimidine DNA sequences as TFOs. Recently a new generation of PNAs called “pseudo-complementary PNAs” (pcPNAs) were synthesized (Izvolsky, K. I. et al., Biochemistry 39: 10908-10913 (2000)). These PNAs target the designated sites on DNA that contain mixed sequence of purines and pyrimidines via double duplex invasion mode. Since the backbone of PNA is not charged, the lack of electrostatic repulsion leads to the formation of strong and stable complexes with DNA. Also, PNA has a smaller mass per monomer unit than DNA and is generally resistant to degradation by enzymes that can attack the phosphate backbone of an oligonucelotide. These and other properties make PNA a very attractive choice for an ATF DNA-binding domain.

In another embodiment, the DNA binding domain may be a peptide analog, such as polyamides, e.g., polypyrroles and polyimidazoles, described in U.S. Pat. No. 5,874,555. These moieties are thought to bind in the minor groove of the DNA helix and the activation observed for ATF using a polyamide DNA binding domain is not as great as for ATFs having a oligonucleotide DNA binding domain. It is thought that the nature of binding of the DNA binding protein within the minor groove of the DNA double strand may limit the ability of the attached transcription effector to interact efficiently with the proteins comprising the transcription machinery.

In still other embodiments, the DNA binding domain is a non-peptidic binding domain including sequence-specific DNA-binding natural products such as antibiotics or other moieties using small organic material.

The effector domain, C, can be a positive (an activator) or a negative (a repressor) modulator of the amount of basal level of transcription (mRNA). Many functional activator and repressor peptides are known and may be used in the ATF molecules of the present invention. In some embodiments, the effector domain includes a suitable chemical coupling group (e.g., an amine, carboxyl or thiol group) that allows for the formation of the covalent bond between the effector and the linker (FIG. 1). In other embodiments, the effector domain, linker and DNA binding domain (or a sub-component thereof) are synthesized as a single molecule, so that no additional chemical coupling group is provided.

In some embodiments, the effector domain is most typically a peptide, selected for its ability to up or down regulate transcription. The peptide may contain either L- or D-amino acids. Exemplary, but non-limiting, examples of activating effectors include VP-16, Oct-2, and active fragments of VP-16 and Oct-2. In at least some embodiments, a 29-mer or a 14-mer of the VP-16 peptide has been shown to have an exceptionally strong activation effect.

In at least some embodiments, shorter peptides that include much, if not all, of the activation function of the longer peptide may be used as a part of the ATF molecule. Shorter peptides may be identified by determining the shortest peptide segment of the activator domain, e.g., the “core” activator, that remains functional in in vitro or in vivo assays in order to minimize the size and mass of the effector domain.

In at least some embodiments of the invention, peptides derived from other general types of activation domains, such as a glutamine rich domain of Oct-2, are used as the effector domain. It has been shown that 18 amino acid peptide SEQ ID NO: 3 (FLFQLPQQTQGALLTSQP) forms a core activation sequence of Oct-2 (Tanaka, M. et al. Mol. Cell. Biol. 14: 6046-6055 (1994); Tanaka, M. & Herr, W. Mol. Cell. Biol. 14: 6056-6067 (1994)). Two or three tandem repeats of this sequence form a functional activation domain. A recent report demonstrates that an 8-amino acid peptide sequence SEQ ID NO: 4 (WAVYELLF) forms a relatively strong activation domain when fused to the protein DNA-binding domain (Frangioni, J. V. et al., Nat. Biotechnol. 18: 1080-1085 (2000)).

In at least some embodiments of the invention, the effectiveness of ATFs is enhanced by combining the minimal activation domains from different activators in a single effector. This takes advantage of cooperative effects in transcriptional activation. For example, two different activators binding the same promoter produce a proportionately stronger effect than each of them acting alone, perhaps by having different classes of activation domains contact different coactivators involved in transcriptional initiation (Blau, J. et al., Mol. Cell. Biol. 16: 2044-2055 (1996); Hampsey, M. & Reinberg, D. Curr. Opin. Genet. Dev. 9: 132-139 (1999)). In at least some embodiments, the core sequences from Oct-2 (SEQ ID NO: 3) and VP16 (SEQ ID NO: 1) activation domains are combined in novel arrangements to maximize their effectiveness.

It is also contemplated that the effector configurations are not limited to natural polypeptide chains. In at least some embodiments, polyglycol spacers are introduced between individual core peptide sequences. This increases the conformational flexibility and the ability to interact with other proteins without significant gain in molecular weight.

In some other embodiments, the lower mass and/or higher potency of activation domains is achieved by the incorporation of nonnatural amino acids that stabilize the secondary structure. For example, short alpha-helical structures can be stabilized with introduction of side chain to side chain lactam bridges. These conformational constraints may lead to even shorter peptides that retain much of the conformation and activity of longer, unmodified polypeptide chains.

In at least some other embodiments of the invention, the effector is selected to repress transcription. Attachment of repressor domains, i.e., synthesis of repressor ATFs, is contemplated. Natural transcriptional factors typically contain activation domains or repression domains, and in some cases they may contain both activation and repression domains (Liu, Y. Z. et al. Nucleic Acids Res. 26: 2464-2472 (1998); Tanaka, M. et al., Mol. Cell Biol. 14: 6046-6055 (1994); Tanaka, M. & Herr, W. Mol. Cell Biol. 14: 6056-6067 (1994)). Transcriptional factors containing repressor domains function in an analogous manner as transcriptional factors containing activator domains, that is, they both need to bring the respective domains in the vicinity of the promoter DNA to exert the effect on RNA transcription (Ptashne, M., “A Genetic Switch: Phage Lambda and Higher Organisms,” supra; Ptashne, M. et al., Nature, supra). As opposed to transcriptional activators, the binding of a transcriptional repressor to the promoter results in the decrease in the levels of RNA transcription. This effect is caused by the interference of transcriptional repressor domains with the assembly of the Polymerase II holoenzyme complex through a variety of different mechanisms. These mechanisms range from rearrangements of a chromatin structure through histone deacetylation to interference with action of activation domains (Renkawitz, R. Trends Genet. 6: 192-197 (1990); Cohen, R. N. et al., Mol. Endocrinol. 14: 900-914 (2000); Busch, K. et al., Mol. Endocrinol. 11: 379-389 (1997)). While the repressor domains are very diverse and share very few common structural features, for the most part their net effect on a target gene transcription is similar.

There are many repression domains that are compatible with the ATF concept, such as repression domains derived from Drosophila transcriptional repressors, even-skipped and Kruppel, as well as from human repressor protein Mad1 (Licht, J. D. et al., Mol. Cell Biol. 14: 4057-4066 (1994); Margolin, J. F. et al., Proc. Natl. Acad. Sci. USA 91: 4509-4513 (1994); Han, K. & Manley, J. L. Genes Dev. 7: 491-503 (1993); Beerli, R. R. et al., Proc. Natl. Acad. Sci. USA 95: 14628-14633 (1998); Eilers, A. L. et al. J. Biol. Chem. 274: 32750-32756 (1999)). Although these repressors possess different mechanisms of action, they all have the following unifying properties: (i) ability to repress a wide variety of promoters and act at a distance from the binding site; (ii) portability (maintaining the function when transferred to a different DNA-binding moiety); and (iii) relatively small size. For example, it has been demonstrated that a 13 amino acid peptide derived from the human protein Mad1 repression domain is able to confer the repression function when fused with a heterologous DNA-binding domain (Eilers, A. L. et. al. J. Biol. Chem., supra). Synthesized peptide sequences are derived from these repressors and may be used to make repressor ATFs substantially as is described and shown herein for activator ATFs.

Transcriptional assays for repression in vitro and in vivo may be performed with templates that, for example, contain both ATF and GAL4 binding sites. The repressor ATFs are tested for their ability to inhibit the transcriptional activation mediated by GAL4-VP16 and other strong activation domains fused to the GAL4 DNA-binding domain. Alternatively, the assays for repression may be performed with transcriptional templates that contain the ATF binding sites incorporated in the promoter having a constitutively high level of basal transcription such as the CMV immediate early enhancer-promoter (Schmidt et al., Mol. Cell. Biol. 10: 4404-4411 (1990)). In this case, the binding of the ATF repressor to the promoter will result in the decrease in RNA transcription, and the presence of additional binding sites for transcriptional activation is unnecessary.

The term “artificial repressor” has been used to indicate the strategy that involves blocking of the binding of transcription factors to the promoter (Maher et al., Biochemistry 31: 70-81 (1992); Maher, J. L. et al., Science 245: 725-730 (1989); and Larsen, H. J. and Nielsen, P. E., Nucleic Acids Res. 24: 458-63 (1996)). This is most often achieved by triple-helix forming oligonucletides (TFOs), peptide-nucleic acids (PNAs) or some other sequence-specific non-peptidic DNA-binding molecule designed to compete with a particular transcription factor for binding to the same site. The term “passive” repressors is used herein to describe these known artificial repressor molecules and their mode of action. The crucial requirement for passive repressor is that its binding site is located very close, or overlapping with the binding site for a transcription factor (or some other protein) that is necessary for the expression of a given gene. Therefore, the passive repression is possible only on native promoters that contain these rare arrangements of overlapping binding sites. In contrast, the attachment of the “active” repressor moiety to the non-peptidic DNA-binding moiety (as described here) will allow for a much greater flexibility and efficiency of transcriptional repression by artificial repressors (repressor ATFs). For example, the binding of repressor ATF anywhere in the promoter results in repression of transcription because the typical repressor domain, like even-skipped or Kruppel (described above), is able to act over a large distance from the binding site. Therefore, the precise location of binding sites for repressor ATFs within the promoter is not critical for their action as with the “passive” repressors mentioned above. For that reason, repressor ATFs allow for the targeting of a much wider variety of genes than passive artificial repressors. In other words, the ATF concept can be described as a novel method for the delivery of “active” transcriptional effectors (activators or repressors) to the promoters in order to regulate the transcription of a target gene in a predictable manner. Since ATFs are completely artificial (synthetic) molecules, the present invention allows for the delivery to the promoters and the testing of a wide variety of completely artificial chemical moieties for the ability to modulate the transcription of the selected (target) gene.

The DNA binding domain and the effector domain are linked, e.g., covalently linked, through the flexible linker, B. The linker of B portion of an ATF herein is of minimum length and maximum flexibility, so that the effector moiety or domain of the ATF, when the DNA binding domain is bound to its recognition site near or in the promoter on a DNA template, is capable of diffusing relatively freely within a minimum distance from the DNA template, and can interact molecularly with a surface of the various components of the transcriptional machinery of the cell, more particularly, with a surface of a protein component of the RNA polymerase II holoenzyme. Alternatively, the effector may interact with other proteins involved in transcriptional regulation such as histones, histone modifying enzymes or other transcription factors. In at least some embodiments of the invention, the linker is of a flexibility and length such that the effector moiety or domain is free to move above the surface of the DNA.

In at least some embodiments, the flexible linker of the present invention is at least 5 Å, 10 Å or 15 Å, or at least 15 Å, or at least 20 Å, or at least 28 Å in length. The length of the linker may be selected to permit accessibility of the effector domain to interaction (binding) with RNA polymerase II holoenzyme or other associated proteins. It is recognized that the length of the linker may vary dependent upon, among other factors, the location and orientation of the DNA binding domain at the DNA template or the chemical composition of the effector domain. The linker may include at least 10, or at least 20, or at least 30 atoms in the chain (backbone) between the two domains, and may include up to 50, or up to 100 atoms.

The linker is comprised mainly of carbons, hydrogen, nitrogen, oxygen, sulfur and phosphorus. Suitable linkers include flexible moieties, such as polyglycols, or other polyalkoxy moieties, or oligomers derived from monomers of nucleotides, natural or non-natural amino acids and lower alkyls, and preferably oxygen-containing organic moieties. The linker is preferably of low molecular weight, chemically inert and water soluble. In at least some embodiments, the linker is an oxygen-containing moiety, which improves hydrophilicity and is generally desirable for drug development.

The linker is a flexible linkage between the DNA binding domain and the effector domain, and is selected such that the linkage between the two domains occurs while the other domains continue to perform their intended functions. The linker is a crucial component of the ATF composition since it enables the optimal geometric configuration and therefore maximizes the potential biochemical activity of the ATFs. The linker moiety is covalently attached to the DNA binding domain and the effector domain. For covalent linkage, various functionalities may be used, such as amides, carbonic acid derivatives, ethers, esters, including organic and inorganic esters, amino, urethane, urea and the like. To provide linking, the particular domain, e.g., the DNA binding domain or the effector domain, may be modified, for example, by oxidation, hydroxylation, substitution, reduction, etc., to provide a site for coupling to the linker. The domains may terminate in a reactive amine, carboxylic acid, hydroxyl, or thiol group, or the like, which are susceptible to conventional peptide and/or oligonucleotide reactions. It will be appreciated that modifications to the domain that do not significantly effect the domain function are preferred. Depending on the domain, there may be a number of sites available for coupling through the linker. Any suitable linker and linker coupling may be used, so long as it does not interfere with the cellular functions of the two domains.

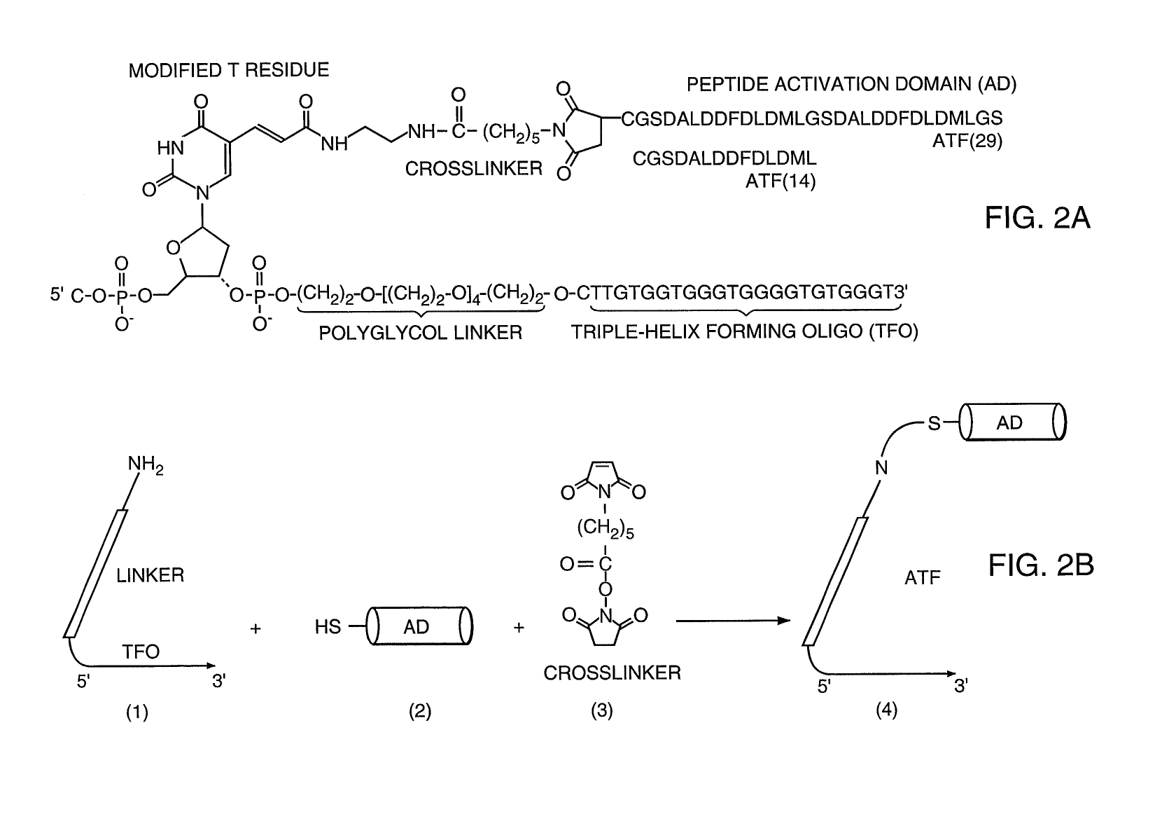

In at least some embodiments, a linker may be readily included in the DNA or PNA strand during synthesis of the DNA or PNA strand in an automated chemical synthesis. One moiety that may be so incorporated is polyglycol spacer. For example, a polyglycol spacer is attached at the end of the DNA binding domain and a reactive group is provided by addition of a modified thymidine bearing a terminal primary amine (See FIG. 2.).

In at least some embodiments, a bifunctional crosslinking agent is used to join the linker to either the DNA binding or effector domains. Suitable crosslinking agents include small bifunctional molecules capable linking two target groups. The target groups typically are the functional groups discussed above. Exemplary thiol-thiol crosslinking groups include dibromobimane. Exemplary amine-amine crosslinking groups include bis(succinimidyl esters), e.g., bis(succinimidyl esters) of 5,5′-dithiobis-(2-nitrobenzoic acid), or ethylene glycol bis(succcinic acid). Exemplary amine-thiol crosslinking agents include amine-reactive maleimide and iodoacetimide derivatives, such as succinimidyl trans-4-(maleimidylmethyl)cyclohexane-1-carboxylate, succinimidyl 3-maleimidylbenzoate, succinimidyl 6-maleimidylhexanoate, or 4-nitrophenyl iodoacetate.

Coupling of amine and carboxylic acid groups may also be facilitated by “zero length” crosslinks, a crosslinking agent that is not incorporated into the final product. Exemplary agents include 1-ethyl-3-(3-dimethylaminopropyl)carbodiimide and 2-ethoxy-1-ethyxocarbony-1,2-dihydroquinoline.

An exemplary ATF is shown in FIG. 2A, which was designed and synthesized using the components depicted schematically in FIG. 2B. A DNA-binding domain was designed based on a modified triple-helix forming oligonucleotide. The 22-base sequence of SEQ ID NO: 5 (5′TTGTGGTGGGTGGGGTGTGGGT3′) binds the corresponding target double stranded sequence by forming a triple-helical complex at physiological pH (Skoog, J. U. et al., Nucleic Acids Res. 21: 2131-2138 (1993)). A long (e.g., greater than 15 Å) and flexible polyglycol linker was introduced to either the 5′ or 3′ end by automated chemical synthesis. This was achieved by synthesizing two molecules of having the base sequence of SEQ ID NO: 6 (5′TTGTGGTGGGTGGGGTGTGGGTXYC3′) and SEQ ID NO: 7 (5′CYXCTTGTGGTGGGTGGGGTGTGGGT3′), and where the X represents the spacer phosporamidite 18 (hexaethyleneglycol spacer, Glenn Research, Sterling, Va. 20164), and the Y represents the thymidine residue bearing the primary amine group on a short tether (Amino-Modifier C6-dT, Glenn Research). This primary amine is able to react with various kinds of chemicals that do not affect the rest of the molecule. This enables the modified residue to serve as an “anchor” for the coupling of various functional domains (“effectors”), the activity of which can than be tested in transcription assays. The effector can be a synthetic peptide, a non-natural peptide (having amino-acids that do not occur in nature), or a non-peptidic organic molecule.

The ATFs of the invention are useful in modulating the expression of a target gene. The target gene can be any gene that is secreted by a cell, so that the encoded product can be made available (or suppressed) at will. Transcription of many genes in vivo are found to be positively or negatively regulated, while the basal level of other genes, often referred to as “housekeeping” genes, can be relatively constant during the lifetime of a cell. Further, regulation of any gene can be specific temporally, only expressed in normal cells at a certain stage of development, or can be tissue specific, only expressed in certain tissue or cell or organ types.

A temporally regulated gene, such as the gene for nestin, or for a metalloproteinase such as a type II collagenase, may be expressed during fetal development in normal cells, but can be up-regulated in a brain tumor or melanoma in the case of nestin, or during metastasis of a tumor in the case of type II collagenase. Alternatively, a fetal gene such as the gene for embryonic hemoglobin, might be turned on to substitute for insufficient adult hemoglobin in a variety of anemia type diseases, such as sickle cell anemia.

For these and many other applications, promoter-specific artificial transcription factors (activators or repressors) are provided, particularly those of low molecular weight capable of acting as drugs, for example, as negative artificial modulators to turn off tumor-specific genes. An ATF useful as a therapeutic drug is of sufficiently low molecular weight to be administered orally, and to permeate a target cell in a subject

Pharmaceutical Compositions.

In another aspect, the present invention provides pharmaceutically acceptable compositions which comprise a therapeutically-effective amount of one or more of the ATF compositions of the present invention, formulated together with one or more pharmaceutically acceptable carrier(s). The pharmaceutical compositions and methods described herein can include one or more ATF compositions of the present invention.

The phrase “therapeutically-effective amount” as used herein means that amount of an ATF composition, or composition comprising such an ATF composition, which is effective for the ATF composition to produce its intended function, e.g., the modulation of gene expression. The effective amount can vary depending on such factors as the type of cell growth being treated or inhibited, the particular type of ATF composition, the size of the subject, or the severity of the undesirable cell growth or activity. One of ordinary skill in the art would be able to study the aforementioned factors and make the determination regarding the effective amount of the ATF composition without undue experimentation.

The phrase “pharmaceutically acceptable” is employed herein to refer to those ATF composition containing such compounds, and/or dosage forms which are within the scope of sound medical judgment, suitable for use in contact with the tissues of human beings and animals without excessive toxicity, irritation, allergic response, or other problem or complication, commensurate with a reasonable benefit/risk ratio.

ATF compositions of the present invention can exist in free form or, where appropriate, in salt form. Pharmaceutically acceptable salts and their preparation are well-known to those of skill in the art. The pharmaceutically acceptable salts of such compounds include the conventional non-toxic salts or the quaternary ammonium salts of such compounds which are formed, for example, from inorganic or organic acids of bases. The compounds of the invention may form hydrates or solvates. It is known to those of skill in the art that charged compounds form hydrated species when lyophilized with water, or form solvated species when concentrated in a solution with an appropriate organic solvent.

The amount of compound which will be effective in the treatment or prevention of a particular disorder or condition will depend in part on the nature of the disorder or condition, and can be determined by standard clinical techniques. In addition, in vitro or in vivo assays may optionally be employed to help identify optimal dosage ranges. Effective doses may be extrapolated from dose-response curves derived from in vitro or animal model test systems. The precise dosage level should be determined by the attending physician or other health care provider and will depend upon well known factors, including route of administration, and the age, body weight, sex and general health of the individual; the nature, severity and clinical stage of the disease; the use (or not) of concomitant therapies; and the nature and extent of genetic engineering of cells in the patient.

The invention also provides a pharmaceutical package or kit comprising one or more containers holding one or more ingredients including a precursor composition having flexible linker covalently bound to a DNA binding domain, the DNA binding domain having affinity for a DNA binding site on a DNA template sufficient to bind the site and modulate the transcription at a promoter precursor composition contains a reactive end group that can be used to couple the precursor compound to a test compound of interest for assessing the activity of the composition in transcription. The kit also includes a transcription mixture comprising a DNA template and a eukaryotic RNA polymerase molecule that forms a complex with the DNA template. Optionally associated with the kit may be instructions for using the precursor composition according to the methods of the invention.

Assays.

The experimental system described herein can be used to test the activation or repression potential of chemical adducts in vitro; however additional embodiments of the invention herein include in vivo assays. The experimental system described herein activates transcription from a linear (typically in in vitro assays) or circular plasmid template that could be used both in in vitro and in vivo assays. For example, the binding of ATF to the promoter DNA is performed in vitro using circular (plasmid) DNA transcription templates, and the resulting pre-formed ATF-template complex is introduced into the tissue culture by the various methods of transformation, including transfection, electroporation, liposome assisted techniques, etc. The quantification of the resulting RNA transcript from a reporter gene transcriptionally downstream from the initiation site (i.e., beta-galactosidase, chloramphenicol acetyl transferase, etc.) in vivo reveals the activity of the tested ATF. Alternatively, a sample of cells that contain a stably incorporated plasmid having the transcriptional template is treated with the ATF. In this case, the binding of the ATF to the corresponding sites in the template promoter occurs in vivo. Being a relatively small, largely non-peptidic molecule, the ATF readily penetrates the cell membranes in an analogous way to penetration by antisense oligonucleotides. Alternatively, ATFs are enclosed in liposomes to assist them in penetrating the cell membrane. Reporter gene activity in cells in the presence and absence of the ATF reveals the extent of transcriptional activation or repression

The ATF of the invention is used in a method for assaying a test composition for activity as a transcriptional modulator. The method includes linking the test compound covalently to a flexible linker domain which is covalently bound to a DNA binding domain to provide a test composition, the DNA binding domain having affinity for a DNA binding site on a DNA template sufficient to bind the site and to modulate transcription at a promoter; contacting the test composition with a transcription mixture including a DNA template, a eukaryotic RNA polymerase molecule capable of forming a complex, either directly or indirectly through other proteins, with the test composition and the DNA template, a buffer and substrates under conditions suitable for RNA synthesis, such that RNA is synthesized; and determining the quantity of the RNA produced in the presence of the test composition compared to a basal level in the absence of the test composition, which is a measure of the activity of the test composition as a ATF composition. In some embodiments, the DNA binding site is a plurality of repeats of the binding site sequence. The synthesized RNA may be quantified using any conventional detection system. Such systems are for quantitation of RNA product are well known in the prior art.

The ATF compositions of the invention could be adapted to develop in vivo screening system for novel ATFs as well as for therapeutic applications as described above (precise regulation of transgenic cells in vivo). For example, instead of transient transfection (as described in in vivo experiments herein below), stable transfected cell lines can be generated with a reporter construct incorporated into the chromosome. Therefore, any new ATF can be tested for the ability to activate or repress this reporter gene. Endogenous genes can also be used as targets, however, in this case, the signal is detected on a DNA arrays (chips).

The high-density DNA and oligonucleotide microarrays (“DNA chips”) allow monitoring the expression of many different genes simultaneously, which allows extension of the in vivo assay because it can provide a number of clues about the effectiveness of a particular ATF design. For example, the monitoring of early changes in gene expression pattern following the treatment of tissue culture cells with ATFs reveals which genes are directly affected by the ATF. ATF targets in genome could be identified without prior knowledge about the sequences in the promoter. The relative levels of gene expression also provides useful information on the activity of a particular effector. In this manner, both DNA-binding and effector domains are characterized in greater detail simultaneously, along with the identification of potential gene targets for possible medical applications in the future. The microarray analysis is not limited to one type of cell; detection kits are commercially available for many different kinds of eukaryotes, from yeast to humans (such as those produced by Affymetrix).

The ATF compositions of the invention may be used in a variety of applications, such as the ones described herein above, and in particular may be used in the gene therapy. In many instances, the ability to switch a therapeutic gene on and off at will or the ability to titrate expression with precision are important for therapeutic efficacy. This invention is particularly well suited for achieving regulated expression of therapeutic target genes in the context of human gene therapy.

The invention is further illustrated in the following examples, which are provided for the purpose of illustration only and are not intended to be limiting of the invention, the full scope of which is shown in the claims that follow the specification.

### EXAMPLES

### Example 1

### Design and Synthesis of the ATF

In these experiments, the effector domains were a series of synthetic peptides derived from the well-studied activation domain of the Herpes simplex viral protein VP 16, one of the strongest transcriptional activators found in nature. These peptides contain the 29 amino acid sequence of SEQ. ID NO: 8 (CGSDALDDFDLDMLGSDALDDFDLDMLGS), which has a cysteine residue attached to two copies of the VP16 amino acid sequence of SEQ. ID NO: 12 SEQ ID NO: 8 including the double copy of the VP16 amino acid retains about 70% of the activity of the full-length VP 16 when fused to the GAL4 DNA-binding domain (Seipel et al., EMBO J. 11: 4961-4968 (1992); and Nyanguile et al., Proc. Natl. Acad. Sci. USA 94: 13402-13406 (1997)). The thiol-bearing cysteine residue is added at the amino terminus where it serves as the attachment point for conjugation to the rest of the ATF molecule.

All peptides were synthesized on an automated peptide synthesizer using standard FMOC chemistry. The chemical conjugation of a peptide and the oligonucleotide was accomplished through the use of the bifunctional crosslinker, N-hydroxy succinimidyl 6-maleimidylhexanoate, obtained from Molecular Probes (Eugene, Oreg.) (see FIG. 2A). This commercially available crosslinker has two functional groups separated by a six carbon chain: an ester of succinic acid reacts specifically with primary amines, and a maleimide functional group that reacts specifically with thiols under certain conditions. The coupling reaction was performed in successive steps. Since the ester group was the more labile of the two, the crosslinker was first coupled to the primary amine in the oligonucleotide. Excess crosslinker was removed by chloroform extraction and ethanol precipitation. The second step involved the reaction between the thiol group in the peptide and the maleimide functional group of the crosslinker. The purification of the conjugate from the excess peptide and the unreacted DNA was accomplished by reverse-phase HPLC. The purified aliquots were dried in a lyophilizer and subsequently dissolved in ultra-pure water (Ambion, Austin, Tex.) and stored at −70° C.

### Example 2

### In vitro Transcription Assays

Two 26-base oligonucleotides having the sequence of SEQ. ID NO: 9 and SEQ. ID NO: 10 were annealed and ligated to yield a series of double-stranded DNA fragments containing multiple binding sites for triple-helix formation. A fragment containing 5 copies of the site as shown in the duplex of SEQ ID NO: 9 and SEQ ID NO: 10 was purified by agarose gel electrophoresis and inserted into the HindIII restriction site in the polylinker of an embodiment of the G5E4T series of transcription templates.

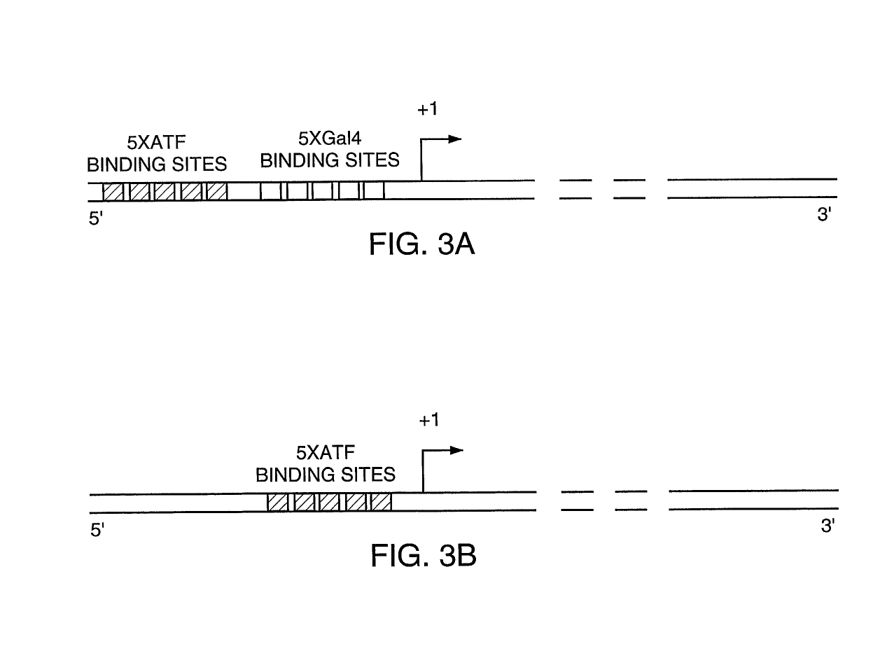

FIG. 3 shows the promoter regions of the transcription templates. In FIG. 3A, the control template contains promoter to modulated transcription five GAL4 and five ATF binding sites incorporated in the promoter at −53 and −155 bp relative to the +1 transcription start site, respectively; and in FIG. 3B the ATF assay template contains five ATF binding sites incorporated at −65 bp.

The resulting plasmids were linearized by digestion with EcoRI, purified and the linear templates were stored in ultra-pure water at −20° C. The transcription assay was initiated by incubation of 200 ng of linearized transcription templates with ATFs in the binding buffer containing 10 mM Tris pH 8, 40 mM MgCl₂ and 100 mM KCl at room temperature for 24 hours. The total volume of binding reaction was 5 microliters. After binding reaction, salt and buffer conditions were adjusted by the addition of 5 microliters of water, 6 microliters of 1×HeLa buffer (20 mM HEPES pH 7.9, 100 mM KCl, 0.2 mM EDTA, 0.5 mM DTT, 20% glycerol) and 5 microliters of HeLa crude nuclear extract (Promega, Madison Wis.). An aliquot of GAL4-VP16 protein (1 microliter total) was added to the control templates and all reactions were pre-incubated for 10 minutes at 30 degrees C. The transcription was initiated by the addition of 1 microliter of a mixture of ribonucleotide triphosphates (ATP, CTP, GTP, 10 mM each) and 0.5 microliters of ³²P-alpha-UTP (NEN, Boston, Mass.). After further incubation at 30° C. for 30 minutes all transcription reactions were terminated by the addition of 100 microliters of stop buffer (0.5 M sodium acetate, 0.2% SDS, 10 mM EDTA, 1 microgram/ml glycogen, 1 microgram/ml Proteinase K). The reactions were vortexed, incubated at 37° C. for 10 minutes, extracted with phenol/chloroform, and precipitated with 250 microliters of ice-cold ethanol. The pellets were dissolved in 20 microliters of standard denaturing formamide/dye mix and were loaded and run on a denaturing 6% polyacrylamide gel.

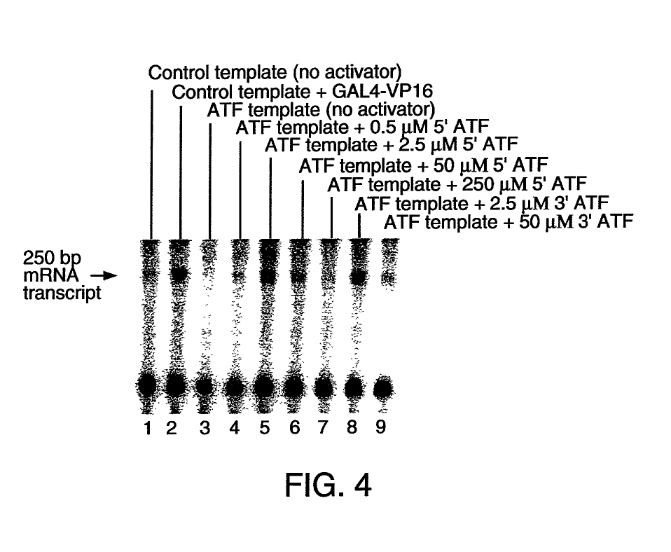

The results are shown in FIG. 4. The lanes 1 and 2 show the activation by the control fusion protein GAL4-VP16 from control templates (FIG. 4A), while all of the other lanes represent the activation by ATFs from the ATF assay template (FIG. 4B). The 250 base transcript initiated at the correct initiation site in each sample is indicated by the arrow. Lane 1 shows the level of basal transcription, in the absence of any transcription factor, from the control template. Lane 2 shows that the addition of the control fusion protein GAL4-VP16 results in marked increase of the quantity of transcription from the control template. Lane 3 shows basal transcription from the ATF assay template. Lanes 4-7 show, as expected, transcription activation as a function of increasing amounts of ATF. Lanes 5, 6, and 7 contain five-fold, 100-fold, and 500-fold more, respectively, ATF than lane 4. The curve shows results in a “bell curve” response. At the optimal concentration of ATF (lane 5), the level of transcriptional activation is equal to, if not higher than, the maximum levels of activation observed from the control template in the presence of control activator protein GAL4-VP16.

The results in FIG. 4 show that the natural activator VP16 fused to the GAL4 binding site to produce the fusion GAL4-VP16, strongly activated the transcription from the control template containing five GAL4 binding sites. The run-off transcript initiated at the +1 transcription start site is 250 bp long (indicated by arrow, FIG. 4). At the same time, ATF(29) is able to activate the transcription from templates containing five ATF binding sites in the promoter (FIG. 4). The activity of ATF(29) is absolutely dependent on the presence of the corresponding binding sites in the promoter as it does not activate transcription from the control templates or other templates lacking ATF binding sites (data not shown). The comparison of RNA transcripts confirms that both GAL4-VP16 and ATF initiate the transcription from the same, “correct” (+1) site in the promoter adjacent to the TATA box. Most unexpectedly, the maximum level of transcriptional activation by ATF(29) was comparable to the level that could be obtained with GAL4-VP16. For example, the quantitation of RNA transcripts reveals that GAL4-VP16 and ATF(29) activate transcription 30-40 fold above basal level (FIG. 4). This result corresponds to previously reported maximal levels of in vitro transcriptional activation by GAL4-VP16 from linearized templates containing 5 GAL4 binding sites (Haile, D. T. & Parvin, J. D. J. Biol. Chem. 274: 2113-2117 (1999)). These levels of activation are obtained with ATF concentrations between 2-40 nM, and further increase in concentration leads to diminishing activation (FIG. 4). This data also indicates another similarity of ATFs with strong natural activators: the ability to “squelch” transcriptional activation, presumably by sequestration of the target components of the transcriptional machinery by excess amounts of strong activators (Ptashne, M. Nature 335: 683-689 (1988)). Both the activation and the squelching ability are completely dependent on the presence of the attached activator domain (AD) peptide because the “truncated” ATFs lacking the AD proved completely inactive.

A. Significance of Fully Functional, Highly Potent ATFs.

While there are many successful drugs that target gene products, thus far there is no effective and practical strategy for pharmaceutical targeting of the gene itself. The manipulation of gene expression at the level of transcription has a great potential for drug development because basic regulatory mechanisms are common for a great majority of eukaryotic genes (Roberts, S. G. Cell Mol. Life Sci. 57:1149-1160 (2000)). In principle, a small-molecule drug designed to modulate transcription of a specific gene can be applied to cause a similar effect on a different gene with only small modifications in its DNA-binding moiety. Therefore, cell permeable drugs able to turn specific genes on or off will form the basis for a universal therapeutic strategy, applicable to many different genes of medical interest.

The progress in this field has been hampered by the fact that designing an effective ATF proved to be a major challenge. For example, a previous attempts to combine synthetic peptides with polyamide DNA binders produced transcriptional activators that bind DNA via a minor groove and exhibit very low activity in in vitro assays (Mapp et al, supra). Since these studies employ only the reporter gene containing G-less cassette, it is not clear whether the overall increase in RNA is due to the specific or non-specific (i.e. random) transcript initiation.

However, to demonstrate the usefulness of ATF as novel tools and develop the first practical applications, it is necessary to achieve a biological activity and specificity to approach that of commonly used natural transcription factors. Results of the present study show for the first time that it is possible to design a fully functional ATF that (i) initiates RNA transcription from the correct site and (ii) possesses biological activity comparable to strong transcriptional activator proteins like GAL4-VP16.

B. Activation Potency Depends on ATF Chemical Structure.

The design of highly potent ATF was achieved through several iterations of molecular design and synthesis. Without being bound by any particular mechanism, the chemical configuration (in particular, length and flexibility) of the linker likely plays a major role in the optimal presentation of the effector to the transcriptional apparatus. Namely, the combination of a long and flexible polyglycol chain with a more rigid and hydrophobic nucleotide crosslinker structure seems be able to mimic the molecular geometry of transcription factors particularly well. In fact, the in vitro transcription assays described in FIGS. 3 and 4 imply that the ATFs could be even more potent transcriptional activators than GAL4-VP16. For example, GAL4-VP16 protein binds to each of the five sites in the promoter as a dimer, while ATF molecules bind the corresponding triple-helix target sites as monomers (Carey, M. et al J. Mol. Biol. 209: 423-432 (1989)). However, five ATF molecules bound to the promoter elicit a similar effect on transcription in vitro compared to ten GAL4-VP16 molecules (FIG. 4). This high potency of ATF molecules may be due to the simple extended chemical structure involving no bulky protein domains that leaves effectors much more exposed to interaction with RNA Polymerase II holoenzyme and/or other proteins. The exposed effector would in effect facilitate the recruitment of the holoenzyme to the promoter and the initiation of RNA transcription. This conclusion is in accord with previous work showing that 14-mer peptide derived from VP16 (FIG. 2A) is inactive when fused to the GAL4 DNA binding domain through recombinant DNA techniques (Seipel et al, supra). In contrast, the present study demonstrated that the same 14 mer peptide sequence shows a remarkable biochemical activity within the context of ATF.

### Example 3

### An ATF Having the Linker Covalently Attached to the 3′ Terminus of the DNA Binding Domain

The ATF in FIG. 2A, which has a strong positive transcriptional effect (FIG. 4, lanes 3-7), has a DNA binding domain A, covalently attached to a linker B, via the 5′ end of the DNA binding-domain. When the polylinker was attached at the 3′ end of the DNA-binding domain, this ATF too had a strong positive transcription effect (FIG. 4, Lanes 8-9). Lane 8 shows transcription mediated by the 3′ A-B-C structured ATF, and lane 9 shows transcription by a five-fold increase in quantity. The amount of 250 bp run-off transcript found in lane 8, compared to that in lane 3 showing basal levels in the absence of any effector for the template, indicates that the covalent linkage of a flexible polylinker to the activation domain provides a configuration which is a powerful ATF. The presence of a linker plays a role in the positive function, when contrasted to other configurations (Kuznetsova et al., supra).

### Example 4

### An ATF Having L-and-D Amino Acids Activating Domains and 3′ and 5′ Linked DNA Binding Domains.

This example shows the versatility of the ATF compositions of the invention and establishes functionality for both L- and D-versions of the activator domain as well as 3′ and 5′ linked DNA-binding domains.

A. Synthesis of ATFs

The introduction of a linker into the TFO was achieved by synthesizing two molecules having the sequence of SEQ ID NO: 6 (for 5′ ATFs) and SEQ ID NO: 7 (for 3′ ATFs) where X represents the Spacer Phosporamidite 18 (Glenn Research) and the Y represents the modified T residue bearing the primary amine group on a short tether (Amino-Modifier C2-dT, Glenn-Research).

The two different forms (L- and D-) of the 29-mer peptide having the sequence of SEQ ID NO.8 amino acids by standard methods (Nyanguile, O. et al., Proc. Natl. Acad. Sci. USA 94: 13402-13406 (1997); Stanojevic, D. et al., Natl. Struct. Biol. 2: 450-457 (1995)). The chemical conjugation of a peptide to the rest of the ATF molecule was accomplished through the use of a bifunctional crosslinker succinimidyl 6-maleimidylhexanoate (EMCS, Molecular Probes) as described above in Example 1.

B. Transcription Assays.

The templates for in vitro transcription were made by inserting 5 direct repeats of a triple helix binding DNA sequence having a sequence of SEQ ID NO: 11 (5′TTCTCCTCCCTCCCCTCTCCCTCTT3′) into the Hind III restriction site in the polylinker of the GnE4T series of transcription templates (Lin, Y. S. et al., Cell 5A: 659-664 (1988)). Plasmids were linearized by digestion with EcoRI and/or Hind III and the binding of ATFs to the template was performed by incubating 20-100 ng of linearized ATF transcription templates with ATFs in the binding buffer containing 10 mM Tris pH 8, 40 mM MgCl₂ and 100 mM KCl at room temperature for 24 hours. Ample incubation time was permitted for optimal complex formation between ATFs and the promoter. Subsequently, an aliquot of diluted GAL4-VP16 protein (1 microliter total) was added to the control transcription templates and all reactions were incubated for 10 minutes at 30 degrees C. The transcription reactions were performed with HeLa crude nuclear extract (Promega) using the standard procedures (Promega protocols). The final RNA transcripts were resolved on 6% denaturing polyacrylamide gel. Dried gels were exposed on both Kodak Biomax MS film and Fuji Phosphoimager plates. The quantitation of signals was performed with Fuji MacBAS software.

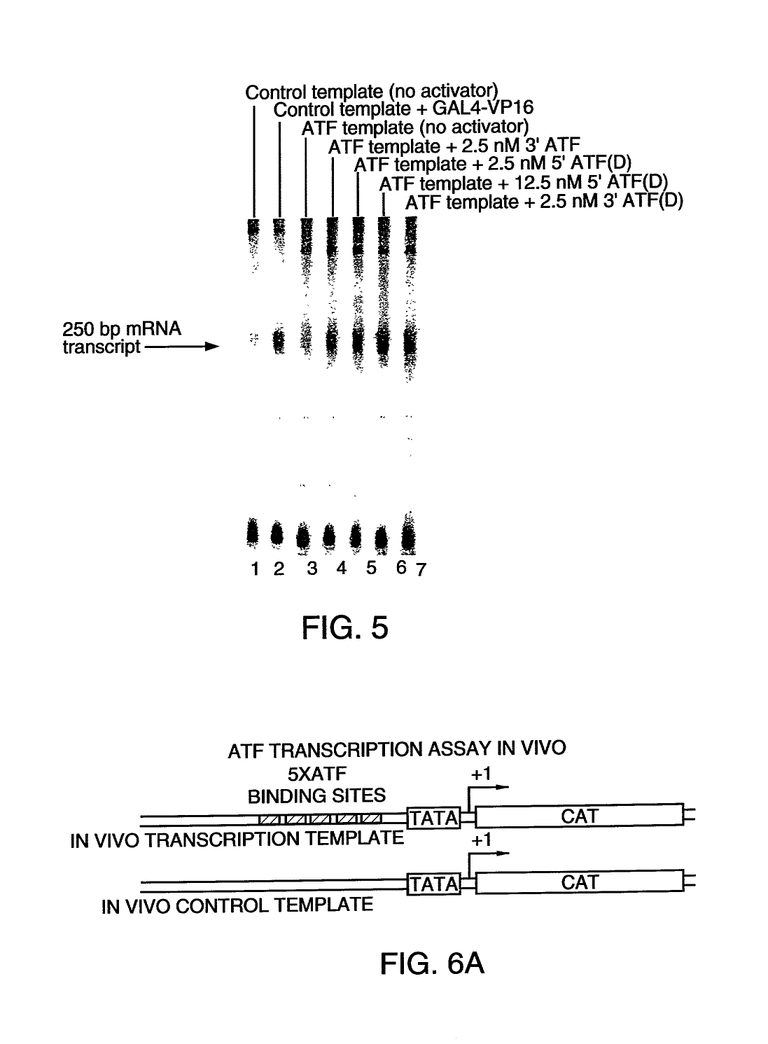

FIG. 5 shows a side-by-side comparison of transcription activation by GAL4-VP16 and the following ATFs: 3′ ATF, 5′ ATF (D) and 3′ ATF (D). Comparison of lanes 2, 4, 5, 6 and 7 indicates that all ATFs are able to activate transcription at least as strongly as GA14-VP 16 in vitro. The comparison of activation by 3′ ATF and GA14-VP16 reveals that 3′ ATF also has a very strong effect on transcription. Despite having slightly different chemical configuration, 5′ ATF and 3′ ATF are very similar in their biological effects, namely, activation strength and the squelching ability. Furthermore, the results in FIG. 5 demonstrate that ATFs synthesized with activation domain consisting of D amino acids are also potent transcriptional activators. It is evident that both 5′ ATF(D) and 3′ ATF(D) activate the transcription from the correct initiation site resulting in a 250 bp RNA transcript that is virtually indistinguishable from those generated by other ATFs or by GA14-VP16. In terms of the activation strength, both 5′ ATF(D) are comparable to other ATFs and to GAL4-VP16.

### Example 5

### In vivo Transcription

A. Synthesis of the ATF.

The ATF molecule was prepared substantially as described in Example 1. The ATF structure included the 22-mer triple-helix forming oligonucleotide (TFO) of SEQ. ID NO: 5, which has been shown to form a stable triple-helical complex with target DNA at physiological pH. A long and flexible polyglycol linker is inserted either near the 5′ end of the TFO. The distal end of the linker bears a modified thymidine residue containing the primary amine group on a short, two-carbon chain tether. This primary amine serves as an anchoring site for the coupling of transcriptional activation domain (AD). The AD consists of a 29-mer (SEQ ID No:8) or 14-mer peptide (SEQ ID NO:12) sequence derived from Herpes simplex viral protein VP16. A thiol-bearing cysteine residue is incorporated at the amino terminus to allow for a covalent linkage with the rest of the ATF molecule. The chemical conjugation of the peptide to the rest of ATF molecule was accomplished through a bifunctional crosslinker. The coupling reaction resulted in two forms of ATF molecule ATF(29) carrying 29 amino acid VP16 peptide, and ATF(14), carrying 14 amino acid VP16 peptide.

B. In vivo ATF Activation

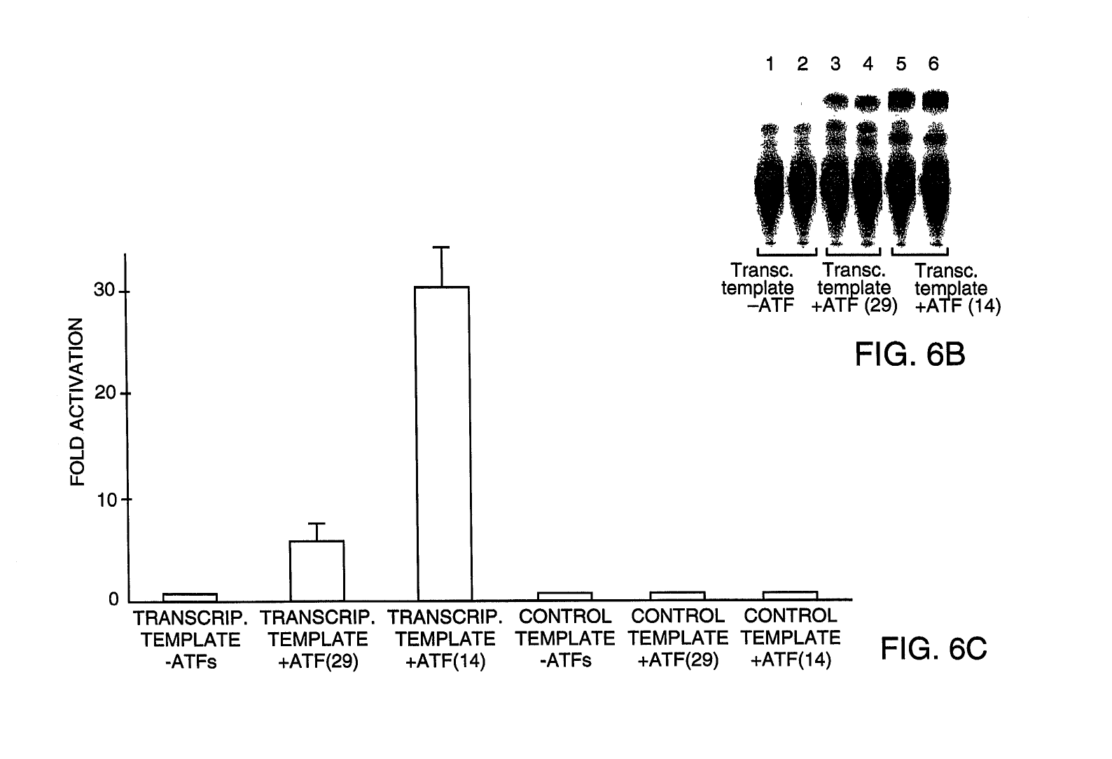

The ability of ATFs to activate transcription in vivo was examined by transient cotransfection assays in BHK-21 tissue culture cells. As a control template, a reporter construct containing the minimal HSV thymidine kinase promoter driving the expression of chloramphenicol acetyl transferase (CAT) reporter gene was used. The transcription template was constructed by incorporating the oligonucleotide with 5 copies of ATF binding sites in between the HindIII and BamHI restriction sites of the polylinker upstream of the control template promoter (FIG. 6A). The transfection mixture contained 1 microgram of the plasmid DNA and 50 nM ATFs. Transfections were performed by using polycationic lipid LipofectAMINE (Life Technologies). Cells were harvested 48 hours after transfection and CAT assays were performed using FAST CAT (Molecular Probes, Inc.) as substrate, and visualized and quantitated on a Fluorimager (Molecular Dynamics). Each reporter construct shown in FIG. 6A was transfected into cells with and without ATF molecules and the activation signal was measured with standard methods.

The results shown in FIGS. 6B and 6C reveal that ATF(29) activates transcription 5-fold above the basal level. At the same time, the ATF(14) caused nearly 30-fold activation of transcription from the same template. This effect is sequence-specific since none of the ATFs were able to activate transcription from the control template lacking the ATF binding sites. Similarly to in vitro assays, the intact ATF structure is necessary for in vivo function as well; namely, ATFs having no peptide attached were not able to activate the transcription from either template.

The results of co-transfection assays in tissue culture cells demonstrate for the first time the substantial biochemical activity of ATFs in an intracellular environment, and further confirm the validity of our design. The ATFs bind to the promoter and elicits a strong (up to 30-fold) and sequence-specific activation of transcription from the reporter gene upon introduction into tissue culture cells (FIGS. 6B and 6C). The apparent ability of ATF(14) to cause a much stronger effect in vivo than ATF(29) is likely due to the differences in cell permeability. Since ATF(14) contains the shorter peptide effector with lower electrostatic charge, it is expected to possess an increased cell permeability compared to the larger ATF(29). The magnitude of transcriptional activation signal obtained with ATFs should be taken in context of the specific experimental set-up. Namely, while in vitro assays allow the direct comparison between synthetic and natural transcription factors, it would be very difficult to interpret such experiments in tissue culture assays. Namely, transfection assays require the introduction of GAL4-VP 16 expression constructs into the living cells, resulting in continuous synthesis of the GAL4-VP16 protein inside the cell. This would be in stark contrast with the introduction of ATF from through the cell membrane, generally at the start of the experiment. Therefore, the calibration of intracellular concentrations of natural vs. synthetic activators over the course of the experiment would be very difficult.

### Example 6

### Methods for Testing Low Molecular Weight Potential Drugs

The efficiency and potency of an embodiment of the ATFs described here, having VP16 amino acid sequences, indicates that by varying the activator chemical moiety, covalently attached as an adduct to the flexible polylinker, in place of the VP16-derived effector domain of the ATF, various chemicals could be tested in transcription assays. These chemicals can include low molecular weight potential drugs, having a molecular weight of less than 3,000 or less than 1,500 daltons. Preliminary results suggest that a wide variety of molecules may be able to mimic the function of activation or repression domains. For example, the 29 amino acid VP16 sequence synthesized using one or more non-naturally occurring D-amino acid shows same activation potential as compared to the sequence made from natural, L-amino acid residues (FIG. 5). Using the methods provided herein, the ability to activate or repress transcription can be shown not to be limited to a moiety having a structure composed of peptides, and that non-peptidic molecules are efficient activation or repression domains.

The non-peptidic nature of the DNA-binding domain of other potential ATFs envisioned herein, along with the linker and small molecule putative effector domains, enables development of smaller, cell-permeable molecules with therapeutic pharmaceutical potential. The chemical structure and composition of the embodiments of the ATF herein could serve as a starting point in development of, or screening for, novel classes of drugs for manipulation of gene expression at the level of transcription.

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed is:

1. A composition for modulating transcription of a gene, comprising:
a non-peptidic DNA binding domain,
a transcriptional effector, one end of the linker being covalently bound to the DNA binding domain, and the other end of the linker being covalently bound to the transcriptional effector, and
a flexible linker.

2. The composition of claim 1, wherein the transcriptional effector activates transcription.

3. The composition of claim 1, wherein the transcriptional effector represses transcription.

4. The composition of claim 1, wherein the transcriptional effector binds a co-effecting protein.

5. The composition of claim 3, wherein the transcriptional repressor binds a histone or histone-modifying protein.

6. The composition of claim 1, wherein the DNA binding domain comprises a nucleic acid.

7. The composition of claim 6, wherein the nucleic acid is a modified nucleic acid.

8. The composition of claim 7, wherein the modified nucleic acid is selected from a group of a nucleic acid having a modified backbone, a nucleic acid having a modified base, and combinations thereof.

9. The composition of claim 7, wherein the linker is bound to 3′ the end or the 5′ end of the nucleic acid.

10. The composition of claim 8, wherein the modified backbone is selected from the group consisting of phosphorothioates and peptide nucleic acids.

11. The composition of claim 1, wherein the DNA binding domain comprises a peptidic nucleic acid.

12. The composition of claim 1, wherein the DNA binding domain comprises a polyamide peptide analog.

13. The composition of claim 1, wherein the DNA binding domain comprises a DNA-binding natural product.

14. The composition of claim 1, wherein the DNA-binding domain comprises an antibiotic or moieties containing small organic material.

15. The composition of claim 1, wherein the DNA binding domain does not contain a plurality of pyrrole or imidazole groups.

16. The composition of claim 1, wherein the transcriptional effector is a polypeptide sequence.

17. The composition of claim 16, wherein the amino terminus of the polypeptide sequence is covalently bound to the linker.

18. The composition of claim 2, wherein the transcriptional activator is a polypeptide sequence, and the polypeptide sequence comprises at least one copy of an activator sequence of amino acids from herpes virus protein VP16 selected from the group consisting of comprising SEQ ID NO:1, SEQ ID NO:2, SEQ ID NO:8 and SEQ ID NO:12.

19. The composition of claim 1, wherein the transcriptional effector is a non-peptidic organic moiety.

20. The composition of claim 1, wherein the transcriptional effector moiety has a molecular weight of less than about 3,000 daltons.

21. The composition of claim 1, wherein the transcriptional effector moiety has a molecular weight of less than about 1,500 daltons.

22. The composition of claim 1, wherein the flexible linker comprises a polyglycol.

23. The composition of claim 22, wherein the flexible linker polyglycol contains at least six glycol units.

24. The composition of claim 1, wherein the flexible linker comprises one or more units selected from the group consisting of nucleotides, peptides, lower alkyls and oxygen-containing alkyl groups.

25. The composition of claim 1, wherein the flexible linker is at least 15 Å in length.

26. The composition of claim 1, wherein the flexible linker is at least 28 Å in length.

27. The composition of claim 1, wherein the amount of transcription initiated on a linear double-stranded DNA template differs by at least ten-fold compared to a second transcription amount initiated in the absence of the composition.

28. The composition of claim 1, wherein the amount of transcription initiated on a linear double-stranded DNA template differs by thirty to fifty-fold compared to the second transcription amount in the absence of the composition.

29. The composition of claim 1, wherein the gene comprises a eukaryotic gene.

30. A composition for activating transcription having the structure A-B-C, wherein A is a triplex-forming nucleic acid, B is a flexible linker, and C is an effector moiety, wherein B is covalently linked to A and C.

31. The composition of claim 30, wherein B is a polyglycol chain, and the covalent linkage of B to C includes a bifunctional crosslinking agent.

32. The composition of claim 30, wherein B comprises a polyglycol which is at least about 28 Å in length, and C comprises an amino acid sequence from herpes simplex virus protein VP16.

33. A method for assaying a test composition for activity as a transcriptional modulator, comprising:
providing a cell comprising a target gene under the control of at least one transcriptional regulatory element;
providing a test composition comprising a DNA binding domain, a test moiety of interest and a flexible linker, one end of the linker being covalently bound to the DNA binding domain, and the other end of the linker being covalently bound to the test compound, the DNA binding domain having affinity for a DNA binding site on a promoter of the target gene sufficient to bind the site;
contacting the cell with a test composition; and
detecting any changes in the level of transcriptional activity of the target gene in the presence of the test composition compared to the level in the absence of the test composition, which is a measure of the activity of the test composition as a transcriptional modulator.

34. A method for assaying a test composition for activity as a transcriptional modulator, comprising:
providing a test composition comprising a DNA binding domain, a test moiety of interest and a flexible linker, one end of the linker being covalently bound to the DNA binding domain, and the other end of the linker being covalently bound to the test compound, the DNA binding domain having affinity for a DNA binding site on a DNA template sufficient to bind the site;
contacting the test composition with a transcription mixture comprising a DNA template, a eukaryotic RNA polymerase molecule capable of forming a complex with the test composition and the DNA template, a buffer and substrates under conditions suitable for RNA synthesis, and a detection system for quantitation of RNA product, such that RNA is synthesized; and,
detecting any changes in the level of transcriptional activity of the target gene in the presence of the test composition compared to the level in the absence of the test composition, which is a measure of the activity of the test composition as a transcriptional modulator.

35. The method of claim 33 or 34, wherein the test moiety is a chemical moiety having a molecular weight of less than about 3 kD.

36. The method of claim 33 or 34, wherein the test moiety is a chemical moiety having a molecular weight of less than about 1.5 kD.

37. The method of claim 33, wherein the test moiety is a cell membrane-permeant.

38. The method of claim 33 or 34, wherein the test moiety is suspected of having transcriptional activation or repressor activity.

39. The method of claim 33 or 34, wherein the changes in transcriptional activity are detected as variations in observed levels of mRNA, or protein product encoded by the target gene.

40. The method of claim 33 or 34, wherein the target gene is selected from the group consisting of a gene encoding a protein conferring resistance to a drug, a gene encoding an enzyme, a gene which rescues an auxotrophic phenotype, and a gene encoding a cell surface antigen.

41. The method of claim 40, wherein the target gene encodes a protein which provides for calorimetric, luminescent or fluorescent detection.

42. The method of claim 33 or 34, wherein the step of providing the test composition is performed using high throughput screening technologies comprising robotized sample distribution into multiwell dishes.

43. The method of claim 33 or 34, wherein the step of detecting changes in transcriptional activity is performed using the high throughput methods of detection wherein automated plate readers have programmable computerized programs for data analysis and display.

44. The method of claim 33 or 34, wherein a plurality of test compositions is provided.

45. A method of altering transcription of a target gene, comprising:
introducing into a cell a composition comprising a DNA binding domain having an affinity for a promoter of the target gene, a flexible linker, and a transcriptional effector, one end of the linker being covalently bound to the DNA binding domain, and the other end of the linker being covalently bound to the transcriptional effector.

46. The method of claim 45, wherein the effector is an activator or a repressor.

47. The method of claim 45, wherein the DNA binding domain is a non-peptidic binding domain.

48. A kit for assaying a test composition for activity as a transcriptional modulator, comprising:
a flexible linker covalently bound to a DNA binding domain, the DNA binding domain having affinity for a DNA binding site on a DNA template sufficient to bind the site and activate transcription at a promoter; and
a transcription mixture comprising a DNA template and a eukaryotic RNA polymerase molecule that forms a complex with the DNA template.

49. The kit of claim 48, wherein the modulator is an activator or a repressor.

50. A composition for modulating transcription of a gene, comprising:
a DNA binding domain,
a transcriptional effector, one end of the linker being covalently bound to the DNA binding domain, and the other end of the linker being covalently bound to the transcriptional effector, and
a non-peptidic flexible linker.

51. The method of claim 45, wherein the transcriptional effector is a polypeptide sequence.

52. The method of claim 51, wherein the amino terminus of the polypeptide sequence is covalently bound to the linker.

53. The method of claim 45, wherein the transcriptional activator is a polypeptide sequence, and the polypeptide sequence comprises at least one copy of an activator sequence of amino acids from herpes virus protein VP 16 selected from the group consisting of comprising SEQ ID NO:1, SEQ ID NO:2, SEQ ID NO:8 and SEQ ID NO:12.

54. The method of claim 45, wherein the transcriptional effector is a non-peptidic organic moiety.

55. The method of claim 45, wherein the transcriptional effector moiety has a molecular weight of less than about 3,000 daltons.

56. The method of claim 45, wherein the transcriptional effector moiety has a molecular weight of less than about 1,500 daltons.

57. The method of claim 45, wherein the flexible linker comprises a polyglycol.

58. The method of claim 57, wherein the flexible linker polyglycol contains at least six glycol units.

59. The method of claim 45, wherein the flexible linker comprises one or more units selected from the group consisting of nucleotides, peptides, lower alkyls and oxygen-containing alkyl groups.

60. The method of claim 45, wherein the flexible linker is at least 15 Å in length.

61. The method of claim 45, wherein the flexible linker is at least 28 Å in length.

62. The method of claim 45, wherein the amount of transcription initiated on a linear double-stranded DNA template differs by at least ten-fold compared to a second transcription amount initiated in the absence of the composition.

63. The method of claim 45, wherein the amount of transcription initiated on a linear double-stranded DNA template differs by thirty to fifty-fold compared to the second transcription amount in the absence of the composition.


SEQUENCE LISTING (12 sequences)

SEQ ID NO: 1 - protein, 11 aa, Artificial Sequence
  synthetic composition useful as artificial transcriptional factor
GSDALDDFDLD

SEQ ID NO: 2 - protein, 23 aa, Artificial Sequence
  synthetic composition useful as artifical transcriptional factor
CGSDALDDFDLDGSDALDDFDLD

SEQ ID NO: 3 - protein, 18 aa, Artificial Sequence
  synthetic composition useful as artificial transcriptional factor
FLFQLPQQTQGALLTSQP

SEQ ID NO: 4 - protein, 8 aa, Artificial Sequence
  synthetic composition useful as artificial transcriptional factor
WAVYELLF

SEQ ID NO: 5 - DNA, 22 bp, Artificial Sequence
  synthetic composition useful as artificial transcriptional factor
ttgtggtgggtggggtgtgggt

SEQ ID NO: 6 - DNA, 24 bp, Artificial Sequence
  synthetic composition useful as artificial transcriptional factor
ttgtggtgggtggggtgtgggtyc

SEQ ID NO: 7 - DNA, 25 bp, Artificial Sequence
  synthetic composition useful as artificial transcriptional factor
cycttgtggtgggtggggtgtgggt

SEQ ID NO: 8 - protein, 29 aa, Artificial Sequence
  synthetic composition useful as artificial transcriptional factor
CGSDALDDFDLDMLGSDALDDFDLDMLGS

SEQ ID NO: 9 - DNA, 26 bp, Artificial Sequence
  synthetic composition useful as artificial transcriptional factor
gttctcctccctcccctctccctctt

SEQ ID NO: 10 - DNA, 26 bp, Artificial Sequence
  synthetic composition useful as artificial transcriptional factor
aagaggagggaggggagagggagaac

SEQ ID NO: 11 - DNA, 25 bp, Artificial Sequence
  synthetic composition useful as artificial transcriptional factor
ttctcctccctcccctctccctctt

SEQ ID NO: 12 - protein, 14 aa, Artificial Sequence
  synthetic composition useful as artificial transcriptional factor
CGSDALDDFDLDML